(19) United States
(12) Patent Application Publication
(10) Pub. No.: US 2015/0020806 A1
(43) Pub. Date: Jan. 22, 2015

(54) DRY POWDER INHALER FOR DELIVERING MULTIPE AGGLOMERATE FORMULATIONS

(71) Applicant: MERCK SHARP & DOHME CORP., RAHWAY, NJ (US)

(72) Inventors: MICHAEL A. GALLUPPI, MIDDLESEX, NJ (US); ALEKSANDR ZUYEV, DENVILLE, NJ (US); ANNANIY BERENSHTEYN, EDGEWATER, NJ (US); SCOTT C. BROWN, PRINCETON, NJ (US)

(21) Appl. No.: 14/332,951

(22) Filed: Jul. 16, 2014

Related U.S. Application Data
(60) Provisional application No. 61/856,256, filed on Jul. 19, 2013.

(51) Int. Cl. A61M 15/00

(52) U.S. Cl. CPC A61M 15/0008; A61M 15/0086; A61M 15/0091; A61M 2202/064
USPC 128/203.15

(57) ABSTRACT

Various embodiments provide drug products and dry powder inhalers. With various embodiments of the present invention, a de-agglomeration chamber and by-pass channel are provided which allow for various de-agglomerating capabilities for dry powder dispensers.

### FIELD OF THE INVENTION

This invention relates to dry powder inhalers, drug products and, more particularly to an arrangement useful for preparing targeted de-agglomeration of dry powders containing single and combination active pharmaceutical ingredient(s) (API) (also known as “active pharmaceutical agent(s) (APA)”) in dry powder inhalers (DPI).

### BACKGROUND

Various devices have been used in order to dispense an inhaled metered dose of APA such as, including pressurized aerosol devices, nebulizers, pump inhalators and the like. There is growing demand for powder dispensing devices, such as DPI's, which can dispense metered doses of powdered medicament. With such devices, the powder is withdrawn by inhalation so there is less need to be concerned with synchronizing release of medication with the exact start of inspiration to insure quality of the product delivery. Additionally, dry powders may be more stable than the liquid compositions that may be found in other inhaler device forms.

The particles containing the API that leave a DPI are desirably within a particular size range that target a specific area of the lung. If the particles containing the API are too large, they may not enter the respiratory tract, but instead, will be deposited in the mouth or pharynx and possibly enter the digestive tract. Desirably, the DPI will deliver a consistent fine particle dose (FPD) to the targeted area of the lung. In the case of combination therapies, which consist of two or more API's, it may be useful to have a DPI be able to provide a different FPD for each of the API's. This is also useful when trying to match the performance of other dry powder inhalers for generic versions of marketed products.

Current DPI's may have a reservoir that holds the powder in the form of agglomerates, otherwise known as agglomerate formulations that contain an API. “Agglomerates”, “agglomerate formulations”, “powder”, and “dry powder” are used interchangeably herein and refer to product at various points in a device, including before and after de-agglomeration as discussed herein. As the device is actuated, the reservoir will meter a dose of agglomerates that contains the API. After the device is actuated, the patient inhales to force the agglomerates to be carried through inhaler flow channels and break up into fine particles. These fine particles will desirably deliver a consistent dose of the API to the targeted lung area of the consumer.

Current designs for DPI's and de-agglomeration techniques are described in U.S. Pat. No. 6,240,918, U.S. Pat. No. 5,829,434, U.S. Pat. No. 5,394,868, and U.S. Pat. No. 5,687,710. Swirl nozzles have also been used to de-agglomerate the dry powder. De-agglomeration in a swirl nozzle can be achieved by introducing changes in direction in flow in a channel such that the powder is forced to strike against various channel wall sections due to the changes in direction.

Current dry powder inhalers may be only capable of delivering a dose or a combination of API's with an overall efficiency. If the fine particle fraction of the dose is low, then the rest of the dose may be undesirably swallowed and absorbed through the digestive tract. Additionally, the total delivered dose of API may be limited due to the fact that only a certain total amount of powder may be dispensed from the current DPI's. Thus, it would be desirable to increase the ability of current DPI's, which may be dependent upon API characteristic(s), to deliver a higher fine particle fraction and fine particle dose and to be able to adjust the fine particle dose of at least one API, including in a combination product, to target different areas of the lung or to match dosing performance of another inhaler or product.

### SUMMARY

In one aspect, a dry powder inhaler is provided herein for delivering multiple agglomerate formulations. In particular, the thy powder inhaler is useful for dispensing inhaled doses of dry powder containing single and combinations of APIs. In one embodiment, the dry powder inhaler includes a de-agglomeration chamber having at least one exit opening, a first inlet opening and a second inlet opening which are arranged such that, upon inhalation of a dose, negative pressure is applied to the exit opening which causes a first stream of dry powder to be entrained from the first inlet opening into the de-agglomeration chamber directed toward the exit opening. The negative pressure also causes a second stream of dry powder to be entrained from the second inlet opening into the de-agglomeration chamber directed toward the exit opening. The first and second streams of dry powder collide in the de-agglomeration chamber to form a collective stream passing through the exit opening. The dry powder inhaler also includes at least one by-pass channel disposed adjacent to, and separated from, the de-agglomeration chamber. The by-pass channel includes an inlet and an outlet. Upon inhalation of a dose, negative pressure is applied to the outlet which causes a third stream of dry powder to be entrained from the inlet directed toward the outlet. The third stream of dry powder does not pass through any portion of the de-agglomeration chamber. Advantageously, with the subject invention, fine particle fractions may be prepared for different components of dry powder in the same dose. For example, with a multipart combination, a finer particle powder may be prepared by directing powder through the de-agglomeration chamber, with larger particles being simultaneously caused to pass through the by-pass channel, with all of the powder ultimately being delivered to a patient as a unit dose. This allows for a targeted approach to drug delivery.

An embodiment of the present invention provides a dry powder inhaler that is capable of providing different fine particle fractions of two or more API's. A device is provided which may utilize three separate flow paths containing the agglomerates. Two of these flow paths may be through the de-agglomeration chamber which collides two of the streams of particles while the third stream of particles may by-pass the de-agglomeration chamber and go optionally into a swirl nozzle.

An embodiment of the present invention provides for a de-agglomeration chamber arrangement that is useful for de-agglomerating dry powder in a powder dispenser during inhalation of a dose of dry powder, the de-agglomeration chamber arrangement including a polyflux collider having a body at least partially encompassing a volume and the exit opening. The first and second inlet openings and the exit opening are in communication with the encompassed volume. A reference plane passes through the centers of the first and second inlet openings with the exit opening being spaced from the reference plane. The center of the exit opening has a reference axis passing therethrough, which is perpendicular to the reference plane and spaced from the centers of the first and second inlet openings. Upon inhalation of a dose, negative pressure is applied to the exit opening which causes a first stream of dry powder containing one type of API to be entrained from the first inlet opening into the encompassed volume and directed towards the exit opening. The negative pressure causes a second stream of dry powder of the same or different API to be entrained from the second inlet opening into the encompassed volume and directed towards the exit opening. The first and second streams of dry powder particles collide in the encompassed volume, with both possible powder-to-surface impaction and powder-to-powder impaction, to form a collective stream passing through the exit opening which then may be combined with the third stream, possibly of a different API. Subsequently, the combined collective streams define the dose of dry powder. The combined streams may optionally flow through a swirl nozzle for additional de-agglomeration prior to delivery.

Other embodiments of the present invention provide a drug product comprising a dry powder inhaler and a dry powder comprising at least one API, wherein the dry powder inhaler comprises at least three reservoirs capable of storing at least one dose of the at least one API, wherein when the dry powder inhaler is actuated, the at least one dose emitted from the at least three reservoirs collides against each other before exiting the dry powder inhaler and the third does not collide with another.

Additional embodiments of the present invention provide a drug product comprising a dry powder inhaler and a dry powder comprising at least three agglomerates comprising at least three API's, wherein the dry powder inhaler comprises at least three reservoirs capable of storing at least three doses of the at least three APIs, wherein when the dry powder inhaler is actuated, the at least two agglomerates collide against each other in a de-agglomeration chamber before exiting the dry powder inhaler and the third does not enter the de-agglomeration chamber.

Additional embodiments of the present invention provide a drug product comprising a dry powder inhaler and dry powder, wherein said dry powder comprises agglomerate formulations containing different API's, wherein the dry powder inhaler comprises three reservoirs capable of storing said agglomerate formulations wherein one agglomerate formulation is stored in two reservoirs associated with a de-agglomeration chamber and another agglomerate formulation is stored in a third reservoir associated with a by-pass channel.

Additional embodiments of the present invention provide a drug product comprising a dry powder inhaler and dry powder, wherein said dry powder comprises agglomerate formulations containing different API's, wherein the dry powder inhaler comprises three reservoirs capable of storing said agglomerate formulations wherein one agglomerate formulation containing one API is stored in two reservoirs associated with a de-agglomeration chamber and another agglomerate formulation containing one different API is stored in a third reservoir associated with a by-pass channel.

Additional embodiments of the present invention provide a drug product comprising a dry powder inhaler and dry powder, wherein said dry powder comprises agglomerate formulations containing different API's, wherein the dry powder inhaler comprises three reservoirs capable of storing said agglomerate formulations wherein one agglomerate formulation containing tiotropium is stored in two reservoirs associated with a de-agglomeration chamber and another agglomerate formulation containing mometasone is stored in a third reservoir associated with a by-pass channel.

In another embodiment the two reservoirs associated with the de-agglomeration chamber may contain the same agglomerate formulations which comprise the same API each.

In another embodiment the two reservoirs associated with the de-agglomeration chamber may contain two different agglomerate formulations which comprise one API each.

In another embodiment the two reservoirs associated with the de-agglomeration chamber may contain agglomerate formulations wherein one reservoir contains multiple agglomerate formulations containing one, two or three API and the other reservoir contains multiple agglomerate formulations containing one, two or three API.

In another embodiment the two reservoirs associated with the de-agglomeration chamber may contain agglomerate formulations wherein one reservoir contains multiple agglomerate formulations containing one, two or three API and the other reservoir contains multiple agglomerate formulations containing one, two or three API.

In another embodiment the two reservoirs associated with the de-agglomeration chamber may contain two different agglomerate formulations wherein one reservoir contains an agglomerate formulation containing one API and the other reservoir contains an agglomerate formulation containing a different API.

In another embodiment the two reservoirs associated with the de-agglomeration chamber may contain two different agglomerate formulations wherein one reservoir contains an agglomerate formulation containing one API and the other reservoir contains an agglomerate formulation containing no API.

In another embodiment the two reservoirs associated with the de-agglomeration chamber may contain agglomerate formulations wherein the agglomerate formulations contain API selected from: tiotropium, mometasone, fluticasone, salmeterol, budesonide, formoterol, albuterol, levalbuterol, terbutaline, metaproterenol, bitolterol, pirbuterol, ipratropium, beclomethasone, triamcinolone, cromolyn, nedocromil, and montelukast or a pharmaceutically acceptable salt thereof.

In another embodiment the two reservoirs associated with the de-agglomeration chamber may contain two different agglomerate formulations wherein the agglomerate formulations contain API selected from: tiotropium, mometasone, fluticasone, salmeterol, budesonide, formoterol, albuterol, levalbuterol, terbutaline, metaproterenol, bitolterol, pirbuterol, ipratropium, beclomethasone, triamcinolone, cromolyn, nedocromil, and montelukast or a pharmaceutically acceptable salt thereof.

In another embodiment the third reservoir associated with the by-pass channel may contain one, two or three agglomerate formulations comprising one, two or three API that are the same or different.

In another embodiment the third reservoir associated with the by-pass channel may contain an agglomerate formulation comprising two API that are the same or different.

In another embodiment the third reservoir associated with the by-pass channel may contain an agglomerate formulation comprising two API that are the same or different and are selected from: tiotropium, mometasone, fluticasone, salmeterol, budesonide, formoterol, albuterol, levalbuterol, terbutaline, metaproterenol, bitolterol, pirbuterol, ipratropium, beclomethasone, triamcinolone, cromolyn, nedocromil, and montelukast or a pharmaceutically acceptable salt thereof.

In another embodiment the third reservoir associated with the by-pass channel may contain one agglomerate formulation comprising one API.

In another embodiment the third reservoir associated with the by-pass channel may contain one agglomerate formulation comprising one API selected from: tiotropium, mometasone, fluticasone, salmeterol, budesonide, formoterol, albuterol, levalbuterol, terbutaline, metaproterenol, bitolterol, pirbuterol, ipratropium, beclomethasone, triamcinolone, cromolyn, nedocromil, and montelukast or a pharmaceutically acceptable salt thereof.

The dry powder comprising the at least one API may be in the form of an agglomerate. The de-agglomeration chamber may include a polyflux collider arrangement that utilizes colliding streams of dry powder to provide desirable de-agglomeration of dry powders. The agglomerate may also include a substance such as lactose or another API. The agglomerate may not include an API. The agglomerate may be a “placebo” agglomerate and not contain an API, wherein the placebo agglomerate is stored in one of the two reservoirs of a de-agglomeration chamber.

These and other features of the invention will be better understood through a study of the following detailed description and accompanying drawings.

### BRIEF DESCRIPTION OF THE DRAWINGS

FIG. 1 is a perspective view of a dry powder inhaler formed in accordance with the subject invention;

FIG. 2 is an exploded view of a portion of a dry powder inhaler formed in accordance with the subject invention;

FIGS. 3 and 4 show a de-agglomeration chamber and by-pass channel arrangement usable with the subject invention;

FIGS. 5A-5B show a swirl nozzle usable with the subject invention;

FIG. 6 is a cross-sectional view taken along line 6-6 of FIG. 5B;

FIGS. 7-7B show reservoir arrangements usable with the subject invention;

FIG. 8 shows a dose plate usable with the subject invention; and,

FIGS. 9-10 are schematics of possible flow patterns of dry powder streams in accordance with the subject invention.

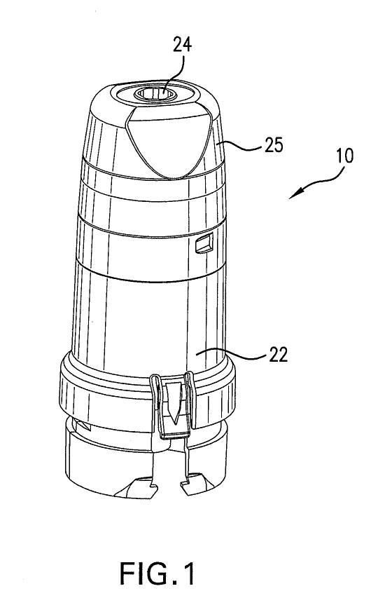

### DETAILED DESCRIPTION

With reference to the Figures, a dry powder inhaler is shown and generally designated with the reference numeral 10. As will be appreciated by those skilled in the art, the dry powder inhaler 10 may be formed with various components and configurations consistent with the disclosure herein. The dry powder inhaler 10 advantageously allows for the delivery of at least one active pharmaceutical ingredient (API) from one or more agglomerates in components of different fine particle fractions or as a multipart combination including dry powder components of different fine particle fractions. This allows for controlled delivery of different components to different portions of a user's respiratory system.

With reference to the Figures, the dry powder inhaler 10 includes a de-agglomeration chamber 12. The chamber 12 includes an enclosed volume 14. At least first and second inlet openings 16, 18 and at least one exit opening 20, preferably one exit opening, are defined in the chamber 12 to be in communication with the enclosed volume 14. The first and second inlet openings 16, 18 and the exit opening 20 are arranged to introduce dry powder into the chamber 12 and cause de-agglomeration thereof. The de-agglomeration may be caused by powder-to-surface impaction and/or by powder-to-powder impaction. Preferably, a combination of both powder-to-surface and powder-to-powder impaction are utilized. More preferably, a powder-to-surface impaction is utilized as an initial impaction. The first and second inlet openings 16, 18 and the exit opening 20 may be arranged in the same manner as the first and second inlet openings and the exit opening of the polyflux collider of U.S. Published Patent Application 2013/0000639, to the assignee herein.

The dry powder inhaler 10 includes a reservoir 22 with at least one chamber for accommodating at least one dose of dry powder, and a nozzle 24, formed on a mouthpiece 25, through which dry powder is administered as a dose in response to a user's inhalation applied thereto. The mouthpiece 25 is formed to be comfortably received in the mouth of a user.

The reservoir 22 may accommodate multiple doses. One or more amounts of dry powder may be metered from the reservoir 22 in any known manner in accordance with a target dose. As discussed further below, the subject invention may utilize three separate streams of dry powder and, thus, may require the metering of three separate amounts of dry powder. By way of non-limiting example, a dose plate 26 may be utilized having a plurality of dose metering holes 28 formed therein. As will be recognized by those skilled in the art, various quantities and geometries may be utilized. The dose plate 26 and the dose metering holes 28 may be configured consistent with U.S. Published Patent Application No. 2012/0304991.

At least one by-pass channel 30 is disposed adjacent to, and separated from, the chamber 12. The by-pass channel 30 includes an inlet 32 and an outlet 34. The by-pass channel 30 defines a flow path completely outside, and separate from, the chamber 12 so that flow of dry powder through the by-pass channel 30 does not pass through any portion of the chamber 12.

In use, upon inhalation of a dose by a user, negative pressure is applied through the nozzle 24 to the exit opening 20 and, separately, to the outlet 34. With negative pressure being applied to the exit opening 20, a first stream of dry powder F1 is caused to be entrained from the first inlet opening 16 into the chamber 12 directed toward the exit opening 20. In addition, the negative pressure also causes a second stream of dry powder F2 to be entrained from the second inlet opening 18 into the chamber 12 directed toward the exit opening 20. As discussed above, the chamber 12 is configured to achieve de-agglomeration of dry powder. The first and second streams F1, F2 preferably collide against a surface to achieve powder-to-surface impaction, such as colliding with a surface, such as top surface 48, in which the exit opening 20 is defined. In addition, with the first and second streams of dry powder being entrained toward the exit opening 20, the first and second streams F1, F2 are caused to collide in the chamber 12 to form a collective stream FS which passes through the exit opening 20. The powder-to-powder collision of the two streams F1, F2 causes de-agglomeration of the dry powder therein. Additional surfaces and/or pathways may cause additional impactions (powder-to-surface and/or powder-to-powder) within the chamber 12, such as including one or more divider(s), as discussed below.

Separately, with negative pressure applied to the outlet 34, a third stream of dry powder F3 is caused to be entrained from the inlet 32 into the by-pass channel 30. By passing through the by-pass channel 30, the third stream of dry powder F3 does not pass through any portion of the chamber 12.

The collective stream FS and the third stream of dry powder F3 are drawn through the nozzle 24 under drawing force of the inhalation. The negative pressure generated by the inhalation is generally applied simultaneously to the exit opening 20 and the outlet 34. The third stream of dry powder F3 may be delivered faster through the nozzle 24 than the collective stream FS due to its more direct (shorter) route to the nozzle 24.

The first, second and third streams of dry powder F1, F2, F3 may be drawn from a single chamber of reservoir 22. In this manner, a dose of dry powder may be delivered having components of the same dry powder but with different fine particle fractions, e.g., the dry powder of the collective stream FS having finer particles (because of the de-agglomeration in the chamber 12) than the third stream of dry powder F3 (which by-passed the chamber 12). With this arrangement, all of the dose metering holes 28 may be supplied by a single chamber of the reservoir 22. As is known in the art, the volume of the dose metering holes 28 may be varied so that the volumes of the various streams of dry powder may be configured as required. For example, the volume of the third stream of dry powder F3 entrained for a dose may be greater than each of the volumes of the first and second streams of dry powder F1, F2 entrained for a dose (with the volume of the collective stream FS for a dose being optionally greater than, equal to, or smaller than the volume of the third stream of dry powder F3).

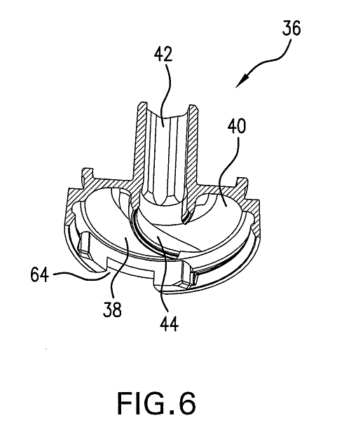

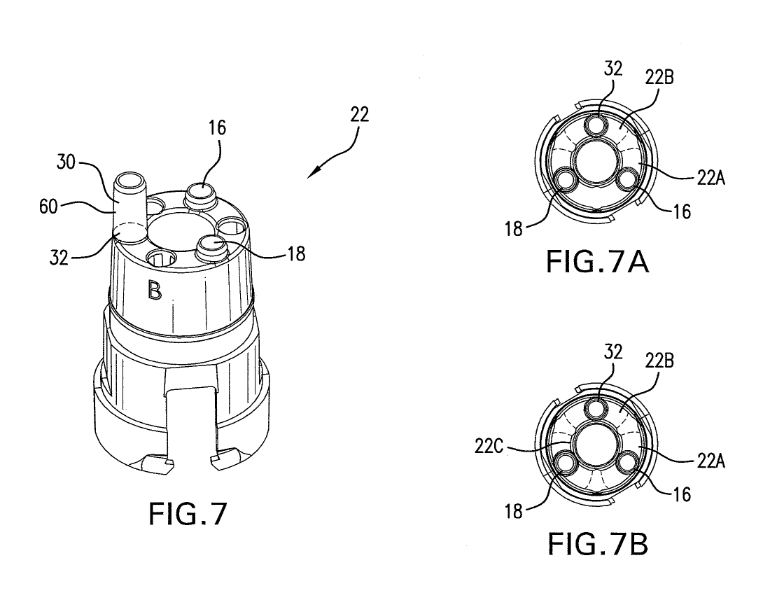

Alternatively, the reservoir 22 may include two or more chambers, including possibly three chambers. For example, the first and second streams of dry powder F1, F2 may be drawn from a first reservoir chamber 22A, while the third stream of dry powder F3 may be taken from a second reservoir chamber 22B, separate from the first reservoir chamber 22A (FIG. 7A). Alternatively, each of the streams of dry powder F1, F2, F3 may be each drawn from an individual reservoir 22A, 22B, 22C so that the dispenser 10 has three separated reservoirs (FIG. 7B). In this manner, multipart combinations may be created administrable by single doses. In addition, as is known, the volume of the dose metering holes 28 may be varied so that the volumes of the various components may be configured as required. For example, the volume of the third stream of dry powder F3 entrained for a dose may be greater than each of the volumes of the first and second streams of dry powder F1, F2 entrained for a dose (with the volume of the collective stream FS for a dose being optionally greater than, equal to, or smaller than the volume of the third stream of dry powder F3 for a dose). As such, with reference to FIG. 8, dose metering holes 28A may be larger (in diameter and/or height) than dose holes 28B and 28C (which, in turn, may be the same or different sizes). The dose metering holes 28B and 28C may be used to dose the first and second streams of dry powder F1, F2, while the dose metering hole 28A may be used to dose the third stream of dry powder F3.

As will be appreciated by those skilled in the art, more than two inlet openings 16, 18 and/or more than one exit opening 20 may be provided with the chamber 12. In addition, more than one chamber 12 and/or more than one by-pass channel 30 may be utilized in various combinations. The chambers 12 may be arranged in parallel and/or in series with by-pass channels 30 being provided to wholly and/or selectively provide by-passing of chamber(s) 12.

Optionally, a swirl nozzle 36 may be provided with the dry powder inhaler 10. The swirl nozzle 36 may provide additional de-agglomeration. As shown in the drawings, the exit opening 20 and the outlet 34 may be positioned to direct the collective stream FS and the third stream of dry powder F3 into an inlet side 38 of the swirl nozzle 36. The swirl nozzle 36, as is known in the art, may be configured to change the direction of flow of the collective stream FS and the third stream of dry powder F3. For example, as shown in the Figures, flow may be deflected by cross-piece 40 and directed towards exit passage 42, at least partially about arcuate wall 44. Re-direction of the flows causes the dry powder to impact various surfaces (e.g., the cross-piece 40, the arcuate wall 44) in causing de-agglomeration thereof. Optionally, the exit passage 42 may be fluted. In this manner, the third stream of dry powder F3 may be subjected to some extent of de-agglomeration and the collective stream FS may be subjected to additional de-agglomeration. It is additionally possible to extend the by-pass channel 30 to also by-pass the swirl nozzle 36 so that the third stream of dry powder F3 does not pass through the swirl nozzle 36 with the collective stream FS being caused to pass through the swirl nozzle 36. Alternatively, the collective stream FS and the third stream of dry powder F3 may be delivered directly to the nozzle 24 with no swirl nozzle being utilized.

During dose delivery, the collective stream FS and the third stream of dry powder F3 may be mixed to some extent prior to final discharge through the discharge opening 24. Mixing of the two streams of dry powder, however, is not required for dose delivery.

With reference to the Figures, the chamber 12 may be defined by body element 46 which may form part of the dry powder inhaler 10. The body element 46 includes a disc-shaped top surface 48 from which depends skirt 50. The skirt 50 may define a part of the outer surface of the dry powder inhaler 10. At least one divider 52 extends between at least two locations interiorly of the skirt 50 so as to separate interior 54 of the body element 46 into at least first and second compartments 56, 58. The chamber 12 may be located in the first compartment 56 and may be partially defined by a portion of the top surface 48, a portion of the skirt 50 and at least a portion of the divider 52. The exit opening 20 may be defined in the top surface 48.

The body element 46 may be positioned adjacent to the reservoir 22 with the first and second inlet openings 16, 18 being defined by the reservoir 22.

The by-pass channel 30 may be located in the second compartment 58, separated from the chamber 12. The outlet 34 may be defined in the top surface 48. The inlet 32 may be defined by the reservoir 22. To constrain the third stream of dry powder F3, the reservoir 22 may define a pipe section 60 which defines the inlet 32 and which extends towards, and, optionally, as shown in the Figures, into communication with the outlet 34 in the top surface 48. The pipe section 60 need not be provided, with the inlet 32 being defined by the reservoir 22 and the by-pass channel 30 being defined by portions of the skirt 50 and the divider 52.

With the use of the body element 46, it is noted that the chamber 12 and the by-pass channel 30 are side-by-side. In addition, preferably, the by-pass channel 30 has the same height as the chamber 12 to be coextensive therewith.

The dry powder inhaler 10 may be assembled, modularly or unitarily (to varying extents), by at least the reservoir 22, the chamber 12, the by-pass channel 30 and the mouthpiece 25. In addition, the swirl nozzle 36 may be utilized. The reservoir 22 may define exterior portions of the dry powder inhaler 10 or may be contained within a body 62 which defines exterior portions of the dry powder inhaler 10.

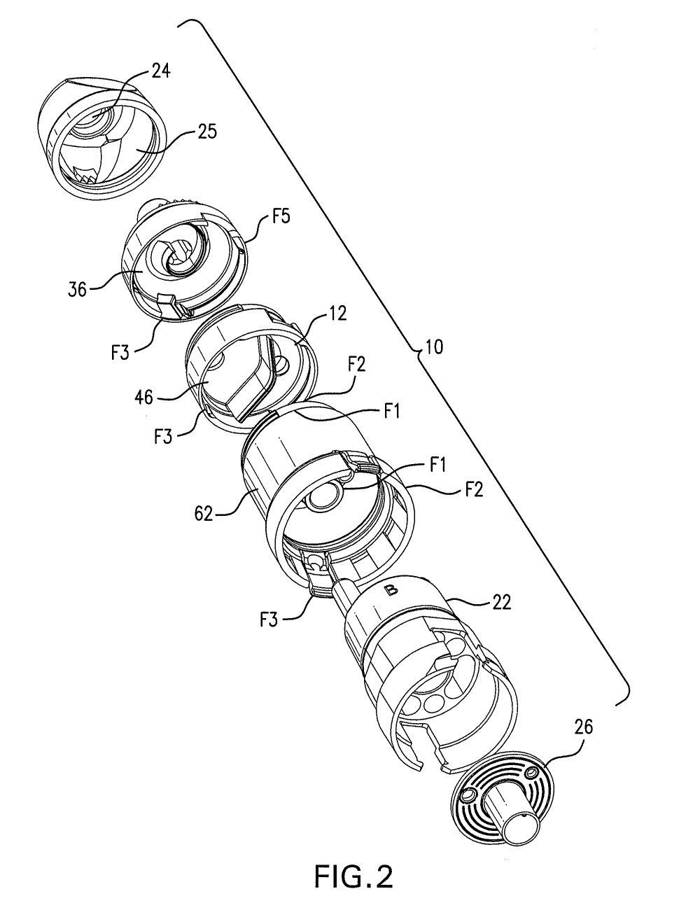

As shown in FIG. 2, during use, the first and second streams of dry powder F1, F2, are caused to be entrained into the chamber 12, while the third stream of dry powder F3 is caused to be entrained into the by-pass channel 30. The collective stream FS and the third stream of dry powder F3 are ultimately drawn to the nozzle 24, optionally through the swirl nozzle 36 if provided.

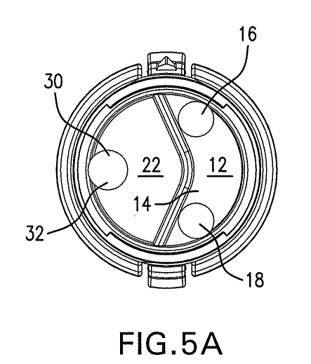

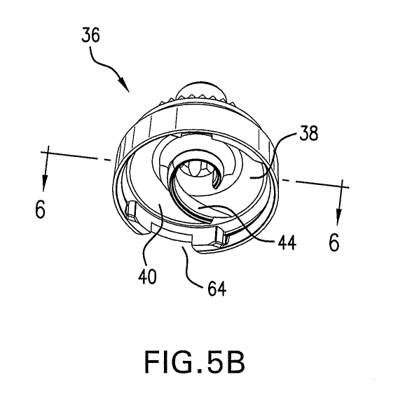

One or more auxiliary inlets 64 may be provided to permit ingress of air into the dry powder inhaler 10, particularly during dose delivery. Preferably, the auxiliary inlet 64 is located between the de-agglomeration chamber 12 and the nozzle 24, such as, for example, in the swirl nozzle 36 (FIGS. 5A-6). The auxiliary inlet 64 allows for a build-up of total volume flow rate and avoidance of undesired deposition of dry powder due to laminar flow conditions.

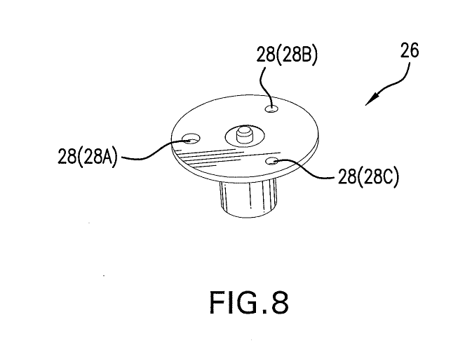

As stated above, the chamber 12 may utilize the polyflux collider arrangement of U.S. Published Patent Application 2013/0000639. By way of non-limiting example, as shown in FIG. 9, the chamber 12 may include a hypothetical reference plane R positioned to pass through the centers C1, C2 of the first and second inlet openings 16, 18. The exit opening 20 is spaced from the reference plane R. In addition, a hypothetical reference axis RA passes through center C3 of the exit opening 20 with the reference axis RA being perpendicular to the reference plane R. The reference axis RA is spaced from the centers C1, C2 of the first and second inlet openings 16, 18. With this arrangement, flow of dry powder coming through the chamber 12 will experience at least two transverse changes in direction. A first transverse change of direction will be experienced upon passing into the encompassed volume 14 through the first or second inlet openings 16, 18. This change of direction may entail a powder-to-surface impaction, e.g., with the top surface 48. A second transverse change of direction will be experienced upon passing from the encompassed volume 14 and through the exit opening 20.

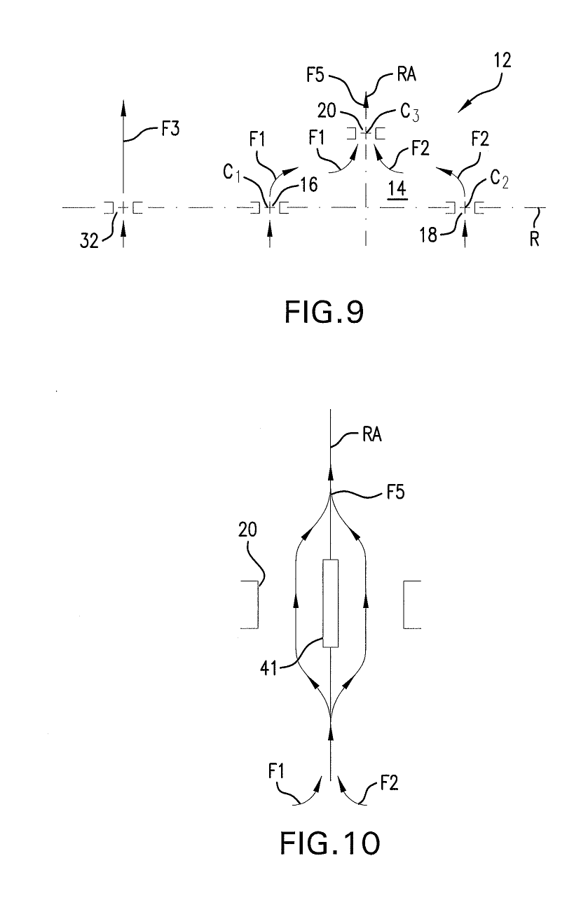

As shown in FIG. 10, another embodiment is exemplified having an arrangement wherein at least one divider 41 spans across at least a portion of, preferably the entirety of, the exit opening 20. The divider 41 may act to guide the flow of dry powder through the exit opening 20. Preferably, the divider 41 is formed as a wall extending in a direction parallel to the intended direction of the flow of dry powder (i.e., in a direction parallel to the reference axis RA). It is further preferred that the divider 41 be configured relative to the exit opening 20 so as to divide the exit opening 20 into symmetrical parts, e.g. being centrally located to divide the exit opening 20 into two symmetrical parts or with a plurality of the dividers 41 being utilized spaced apart to divide the exit opening 42 into a plurality of equal parts. This arrangement permits for the divider 41 to divide the flow into generally equal portions while flowing past the divider 41.

The at least one API may be in the form of an agglomerate. Agglomerates of drug alone or with another substance may be utilized, such as those agglomerates described in U.S. Pat. No. 6,503,537. Any method of agglomerating the solid binder and the APA may be used. Useful agglomerating methods include those which can be accomplished without converting the amorphous content of the solid binder to a crystalline form, prematurely, and which does not require the use of additional binder, can be practiced in accordance with the present invention.

Useful agglomerates include agglomerates ranging in size from between about 100 to about 1500 μm. The agglomerates may have an average size of between about 300 and about 1,000 μm. Useful agglomerates may have a bulk density which ranges from between about 0.2 to about 0.4 g/cm³ or between about 0.29 to about 0.38 g/cm³.

It is useful to have a tight particle size distribution. In this context, particle size refers to the size of the agglomerates. Preferably, no more than about 10% of the agglomerates are 50% smaller or 50% larger than the mean or target agglomerate size. For example, for an agglomerate of 300 μm, no more than about 10% of the agglomerates will be smaller than about 150 μm or larger than about 450 μm.

A useful method of preparing the agglomerates is described in U.S. Pat. No. 6,503,537, which is incorporated herein. Suitable methods involve mixing preselected amounts of one or more pharmacologically active agent(s) and the micronized, amorphous content containing, dry solid binder in a ratio of between about 100:1 and about 1:500; between about 100:1 and about 1:300 (drug:binder); between about 20:1 to about 1:20 or a ratio of about 1:3 to about 1:10 relative to the amount of the solid binder.

Useful agglomerates may have a strength which ranges from between about 50 mg and about 5,000 mg and most preferably between about 200 mg and about 1,500 mg. The crush strength was tested on a Seiko TMA/SS 120C Thermomechanical Analyzer available from Seiko Instruments, Inc. Tokyo, Japan, using procedures available from the manufacturer. It should be noted that strength measured in this manner is influenced by the quality and extent of the interparticulate crystalline bonding described herein. However, the size of the agglomerates also plays a role in the measured crush strength. Generally, larger agglomerates require more force to crush than do the smaller particles.

Various APIs may be utilized. Suitable at least one APIs include but are not limited to an anticholinergic, a corticosteroid, which includes a glucocorticoid, a long acting beta agonist, short acting beta agonist, a dual pharmacology muscarinic antagonist/β2 agonist (MABA), a phosphodiesterase IV inhibitor, a Syk inhibitor, a Jak inhibitor, and a leukotriene receptor antagonist. Suitable medicaments may be useful for the prevention or treatment of a respiratory, inflammatory or obstructive airway disease. Examples of such diseases include asthma or chronic obstructive pulmonary disease.

Suitable anticholinergics include umeclidinium bromide (UMEC), (R)-3-[2-hydroxy-2,2-(dithien-2-yl)acetoxy]-1-1[2-(phenyl)ethyl]-1-azoniabicyclo[2.2.2]octane, glycopyrrolate, ipratropium bromide, oxitropium bromide, atropine methyl nitrate, atropine sulfate, ipratropium, belladonna extract, scopolamine, scopolamine methobromide, methscopolamine, homatropine methobromide, hyoscyamine, isopriopramide, orphenadrine, benzalkonium chloride, tiotropium, tiotropium bromide, and GSK202405, or an individual isomer of any of the above or a pharmaceutically acceptable salt or hydrate of any of the above, or a combination of two or more of the above.

Suitable corticosteroids includes mometasone furoate; beclomethasone dipropionate; budesonide; ciclesonide; fluticasone; fluticasone furoate; fluticasone propionate; dexamethasone; flunisolide; triamcinolone; (22R)-6.alpha.,9.alpha.-difluoro-11.beta.,21-dihydroxy-16.alpha.,17.alpha. -propylmethylenedioxy-4-pregnen-3,20-dione, tipredane, GSK685698, and GSK799943 or a pharmaceutically acceptable salt or hydrate of any of the above, or a combination of two or more of the above.

Suitable long acting beta agonist include carmoterol, indacaterol, TA-2005, salmeterol, vilanterol, olodaterol, and formoterol, or a pharmaceutically acceptable salt or hydrate of any of the above, or a combination of two or more of the above. Suitable short acting beta agonist include albuterol, terbutaline sulfate, bitolterol mesylate, levalbuterol, metaproterenol sulfate, and pirbuterol acetate or a pharmaceutically acceptable salt or hydrate of any of the above, or a combination of two or more of the above. Suitable dual pharmacology muscarinic antagonist/β2 agonist (MABA) include GSK's GSK961081 and Almirall's LAS190792.

Suitable phosphodiesterase IV inhibitors include cilomilast, roflumilast, tetomilast, and 1-[[5-(1(S)-aminoethyl)-2-[8-methoxy-2-(trifluoromethyl)-5-quinolinyl]-4-oxazolyl]carbonyl]-4(R)-[(cyclopropylcarbonyl)amino]-proline, ethyl ester or a pharmaceutically acceptable salt or hydrate of any of the above, or a combination of two or more of the above.

Suitable Syk inhibitors include but are not limited to those disclosed in the following patent applications: WO2011/075515, WO2011/075560, WO2012/154519, WO2012/154518, WO2012/154520, WO2012/151137, WO2013/052394, WO2013/052391, and WO2013/052393.

Suitable Jak inhibitors include but are not limited to those disclosed in the following patent applications: WO 2011/137022, WO 2013/052355, WO 2012/054364, WO 2013/043964, WO 2013/043962, WO 2013/040863, WO 2013/041042, and WO 2003/011285.

Suitable leukotriene receptor antagonists include, among others, montelukast.

Preferred combinations of active pharmaceutical agents include: mometasone/tiotropium; fluticasone/vilanterol; budesonide/formoterol; mometasone/formoterol; ipratropium/albuterol; and fluticasone/salmeterol.

In certain embodiments of the present invention the at least one API includes a corticosteroid, such as mometasone furoate. Mometasone furoate is an anti-inflammatory corticosteroid having the chemical name, 9,21-Dichloro-11(beta), 17-dihydroxy-16(alpha)-methylpregna-1,4-diene-3,20-dione 17-(2 furoate). It is practically insoluble in water; slightly soluble in methanol, ethanol, and isopropanol; soluble in acetone and chloroform; and freely soluble in tetrahydrofuran. Its partition coefficient between octanol and water is greater than 5000. Mometasone can exist in various hydrated, crystalline and enantiomeric forms, e.g., as a monohydrate.

### EXAMPLES

Tests were conducted to evaluate the efficacy of the de-agglomeration chamber/by-pass channel combination (“hybrid polyflux collider arrangement”). All tests utilized Mometasone Furoate and Tiotropiom Bromide in dry powder agglomerate form. In two tests, the Mometasone Furoate and Tiotropiom Bromide were administered in separate doses, while, the test of the hybrid polyflux collider arrangement administered both drugs combined in a single dose. Also, all tests were conducted on an Andersen design cascade impactor, such as that sold by the Thermo Scientific division of Thermo Fisher Scientific, Inc. of Waltham, Mass. at 60 l/min with a test interval of 2 seconds.

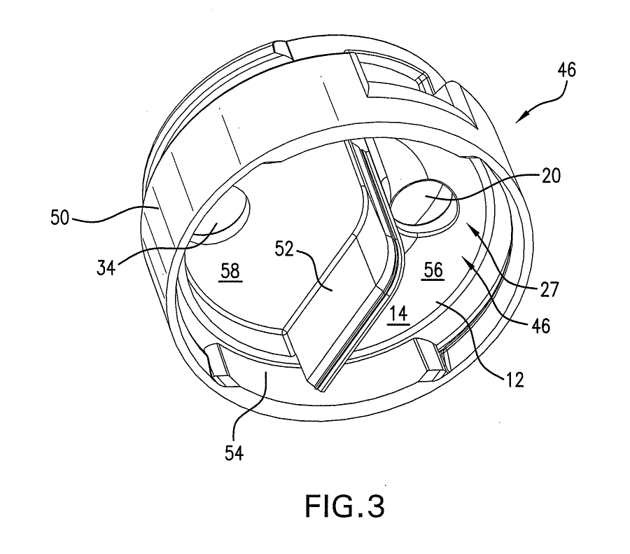

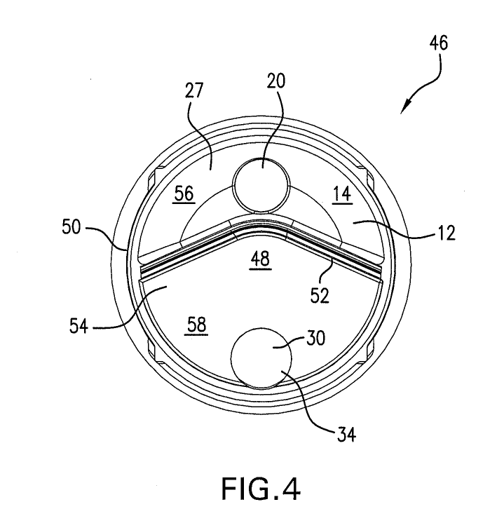

With respect to Table 1, a hybrid polyflux collider arrangement, as shown in FIGS. 3 and 4 herein, was tested with a single dose containing approximately 200 μg of dry powder of Mometasone Furoate and approximately 13 μg of dry powder of Tiotropium Bromide. The test was conducted with simulated inhalation. It is most desirable to recover a maximum level of dry powder at fine particle size (taken to be equal to or smaller than 5 μm).

With the hybrid polyflux collider arrangement of FIGS. 3 and 4 herein, a fine particle fraction (% FPF) of approximately 34% Mometasone Furoate and 38% Tiotropium Bromide was achievable. This correlates to a fine particle dose (FPD) experienced by a patient of approximately 59 μg of Mometasone Furoate and 5 μg of Tiotropium Bromide.

TABLE 1

| | Twisthaler | | Polyflux Collider | | Hybrid Polyflux | |
|---|---|---|---|---|---|---|
| | (Separate | | Twisthaler | | Collider Twisthaler | |
| | Dosing) | | (Separate Dosing) | | (Combined Dosing) | |
| | FPD | | FPD | | FPD | |
| | (mcg) | % FPF* | (mcg) | % FPF* | (mcg) | % FPF* |
| Mometasone | 74 | 38% | 99 | 57% | 59 | 34% |
| Furoate | | | | | | |
| Tiotropium | 3.5 | 26% | 4.1 | 36.4 | 5 | 38% |
| Bromide | | | | | | |

*FPF is the percentage of the total recovered dose under 5 μm

Table 1 also shows data for a “Polyflux Collider Twisthaler”. Tests were done here to dose approximately 200 μg of dry powder of Mometasone Furoate and to dose separately approximately 13 μg of dry powder of and Tiotropiom Bromide using a “Twisthaler” brand inhaler (Merck Corp.) modified with a polyflux collider arrangement as shown in U.S. Published Patent Application 2013/0000639.

Table 1 also shows data for a control test where no polyflux collider arrangement or hybrid polyflux collider arrangement were used. An unmodified “Twisthaler” brand inhaler was used to separately dose approximately 200 μg of dry powder of Mometasone Furoate and approximately 13 μg of dry powder of Tiotropiom Bromide.

In comparing the test results, it can be seen that the hybrid polyflux collider arrangement provides a higher amount of a fine particle dose for the Tiotropium Bromide but a lower fine particle dose for the Mometasone Furoate, which was the desired outcome.

The descriptions of the embodiments of the invention have been presented for purpose of illustration and description. They are not intended to be exhaustive or to limit the invention to the precise forms disclosed, and obviously many modifications and variations are possible in light of the above teaching. The term ‘comprising’ is defined as ‘including but not limited to’.

Percentages are expressed on a weight basis, unless the context clearly indicates otherwise. The mention of any specific drug substance in this specification or in the claims is intended to encompass not only the base drug, but also pharmaceutically acceptable salts, esters, hydrates and other forms of the drug. Where a particular salt or other form of a drug is mentioned, it is contemplated that other salts or forms can be substituted.

What is claimed is:

1. A dry powder inhaler for dispensing inhaled doses of dry powder, the dry powder inhaler comprising:
a de-agglomeration chamber having at least one exit opening, a first inlet opening and a second inlet opening, said at least one exit opening, said first inlet opening and said second inlet opening being arranged such that, upon inhalation of a dose, negative pressure is applied to said at least one exit opening which causes a first stream of dry powder to be entrained from said first inlet opening into said de-agglomeration chamber directed toward said at least one exit opening, said negative pressure also causing a second stream of dry powder to be entrained from said second inlet opening into said de-agglomeration chamber directed toward said at least one exit opening, said first and second streams of dry powder colliding in said de-agglomeration chamber to form a collective stream passing through said exit opening; and,
at least one by-pass channel disposed adjacent to, and separated from, said de-agglomeration chamber, said at least one by-pass channel having an inlet and an outlet, wherein, upon inhalation of a dose, negative pressure is applied to said outlet which causes a third stream of dry powder to be entrained from said inlet directed toward said outlet, said third stream of dry powder not passing through any portion of said de-agglomeration chamber.

2. A dry powder inhaler as in claim 1 further comprising a swirl nozzle having an opening in communication with said outlet of said by-pass channel.

3. A dry powder inhaler as in claim 2, wherein said at least one exit opening of said de-agglomeration chamber is in communication with said opening of said swirl nozzle.

4. A dry powder inhaler as in claim 1 further comprising a nozzle.

5. A dry powder inhaler as in claim 4, wherein said collective stream and said third stream of dry powder are drawn into said nozzle upon inhalation of a dose.

6. A dry powder inhaler as in claim 1 further comprising at least one reservoir chamber for accommodating dry powder.

7. A dry powder inhaler as in claim 6, wherein a first reservoir chamber is provided arranged to supply dry powder to at least said first inlet opening of said de-agglomeration chamber.

8. A dry powder inhaler as in claim 7, wherein a second reservoir chamber, separate from said first reservoir chamber, is provided arranged to supply dry powder to said inlet of said by-pass channel.

9. A dry powder inhaler as in claim 8, wherein a third reservoir chamber, separate from said first and second reservoir chambers, is provided, and wherein, said first reservoir chamber is arranged to supply dry powder to said first inlet and said third reservoir chamber is arranged to supply dry powder to said second inlet.

10. A dry powder inhaler as in claim 1, wherein said negative pressure is applied generally simultaneously to said at least one exit opening and to said outlet.
